(19) United States
(12) Patent Application Publication
(10) Pub. No.: US 2009/0216147 A1
(43) Pub. Date: Aug. 27, 2009

(54) Device For Measurement Of Physiological Signals Of An Object

(75) Inventor: Leenderd Anne Van Eijkern, Zuidhom (NL)

Correspondence Address: JENSEN + PUNTIGAM, P.S., 2033 6th Ave, Suite 1020, SEATTLE, WA, 98121, US

(73) Assignees: PELVISION, Delft (NL); MASSOLT HOLDING B.V., Heerenveen (NL)

(21) Appl. No.: 11/997,977

(22) PCT Filed: Jul. 19, 2006
(86) PCT No.: PCT/NL2006/000375

§ 371 Date: Jun. 25, 2008

(30) Foreign Application Priority Data
Aug. 5, 2005 (NL) NL1029682

(51) Int. Cl. A61B 5/04

(52) U.S. Cl. 600/547; 600/300

(57) ABSTRACT

The present invention relates to a device for measuring physiological signals of an object, for instance a human body of a patient. The device is provided with measuring means for measuring the physiological signals and with signal processing means for at least partially processing the measured physiological signals. According to the invention the device (1) comprises a first housing part (2) to which the measuring means (4A-4F) are attached. The device also comprises a second housing part (3) in which the signal processing means are accommodated. The housing parts (2, 3) are electrically connectable by means of a releasable coupling.

The present invention relates to a device for measuring physiological signals of an object, which device is provided with measuring means for measuring the physiological signals and with signal processing means for at least partially processing the measured physiological signals.

A device of the type stated in the preamble is known in the field. The known device is used to measure diverse types of electrophysiological signals in a human body. These electrophysiological signals are also referred to in the field as ExG signals, and include EEG signals, EOG signals, ECG signals and so on. The known device is provided with a number of electrodes as measuring means, wherein the number of electrodes required depends on the intended measurement. The required electrodes must therefore be connected manually prior to a measurement. Errors are however regularly made in practice during connection of the electrodes. Such an erroneously connected electrode has a considerable negative effect on the signal-to-noise ratio.

The invention has for its object to provide a device of the type stated in the preamble which obviates this drawback.

The device according to the invention has for this purpose the feature that the device comprises a first housing part to which the measuring means are attached, and that the device comprises a second housing part in which the signal processing means are accommodated, wherein the housing parts are connectable by means of a releasable coupling.

The invention is based on the insight of arranging the measuring means on a first releasable part of the housing such that the connection of the measuring means no longer has to be carried out directly by the user, but takes place automatically by means of coupling the first housing part to a second housing part of the device. The measuring means are hereby always connected correctly. According to the invention there can be provided for each type of measurement a separate first housing part having the number of measuring means adapted to the intended measurement. The first housing part is also highly suitable for use as disposable article, i.e. for once-only used on a patient. This guarantees optimum hygiene for every patient.

In a first preferred embodiment the measuring means comprise two or more electrodes which are adapted to measure electrophysiological signals in a human body.

The signal processing means preferably comprise at least one reference amplifier with which the signal-to-noise ratio can be greatly improved.

According to a practical preferred embodiment, the releasable coupling comprises a snap connection for the housing parts with which the connection can be effected in rapid and reliable manner.

In a further preferred embodiment the releasable coupling also comprises an electrical connection for the measuring means and the signal processing means.

In another advantageous preferred embodiment the second housing part comprises test means for testing the operation of the measuring means of a coupled first housing part. The second housing part optionally comprises means for switching off measuring means which are not functioning or not functioning properly. And optimum signal-to-noise ratio is ensured in effective manner by preventing measuring means which are not functioning properly from being able to cause interference.

Optimal convenience of use is provided by a further preferred embodiment in which the first housing part comprises first identification means with information relating to the type of physiological measurement for which the first housing part is adapted, and in which the second housing part comprises second identification means adapted to read the information. The device according to the invention is thus automatically prepared for the intended measurement in optimal manner.

The second housing part preferably comprises means for wireless communication with a further device. These can for instance be utilized in advantageous manner to retrieve software from a further device for the purpose of controlling one or more means of the second housing part.

The invention will now be discussed in more detail with reference to the drawings, in which

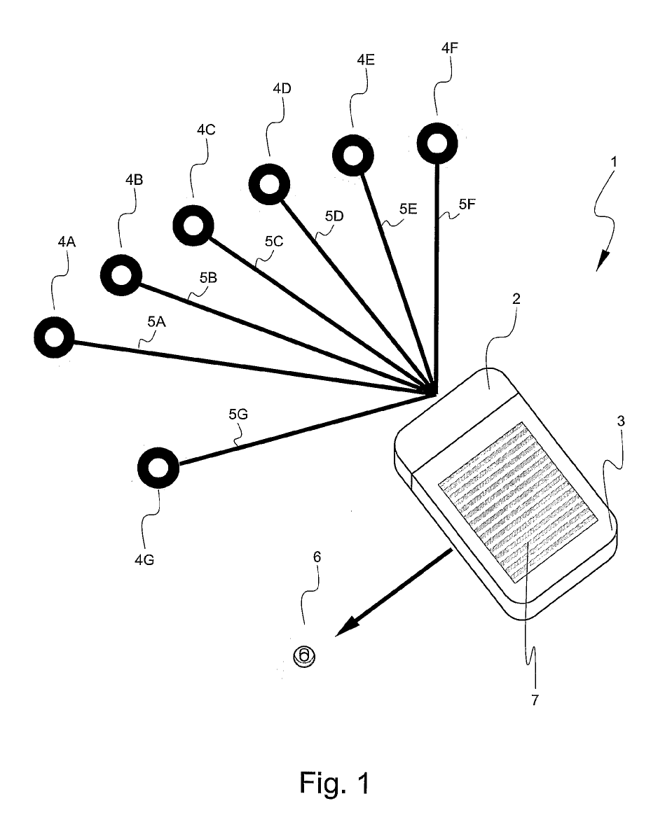

FIG. 1 shows a schematic view of a preferred embodiment of a device according to the invention;

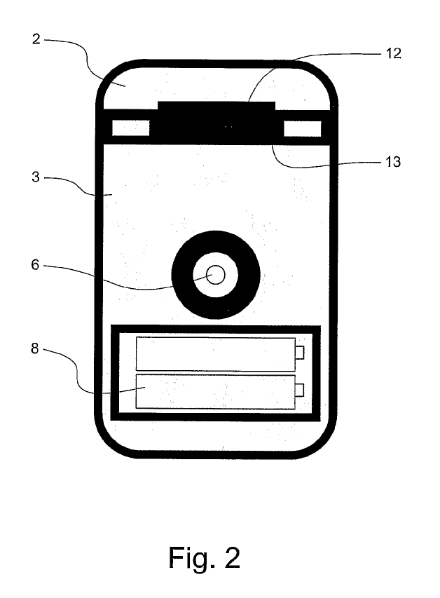

FIG. 2 shows a partly cut-away view of the preferred embodiment of FIG. 1 without measuring means;

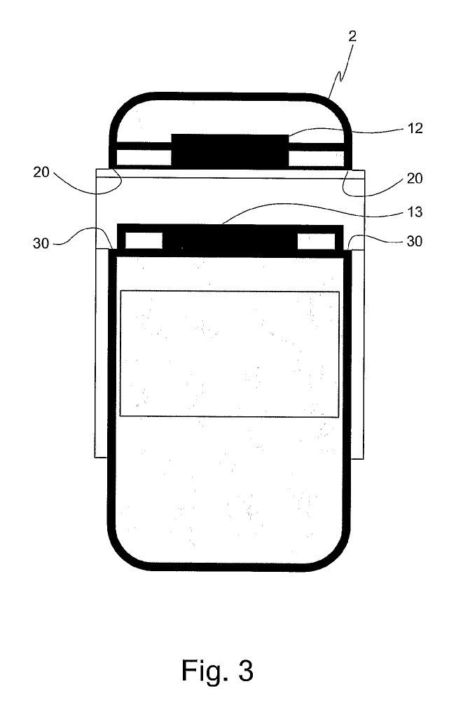

FIG. 3 shows the preferred embodiment of FIG. 1 with exploded parts; and

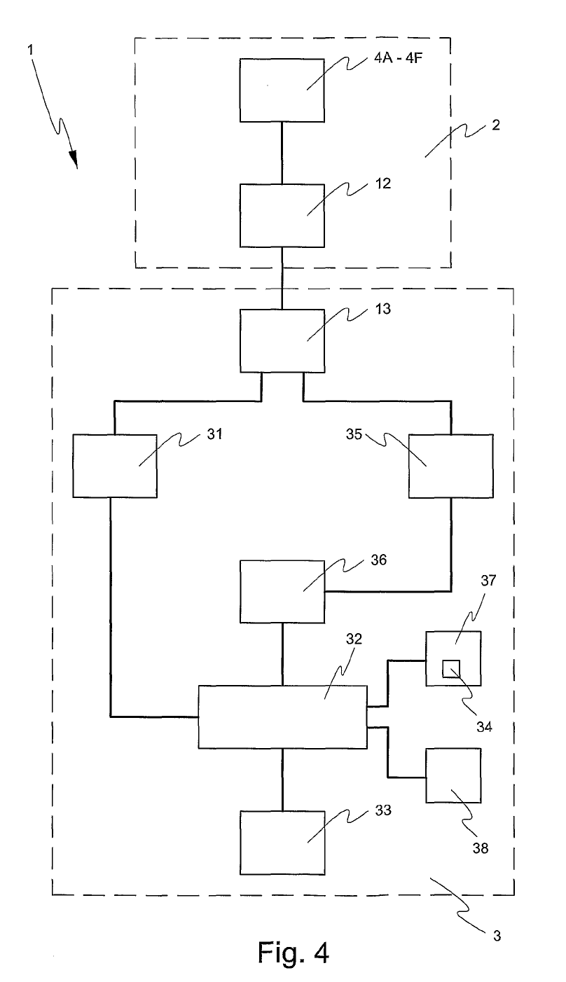

FIG. 4 shows a block diagram for the purpose of illustrating the operation of the device according to the invention.

FIG. 1 shows schematically a device 1 according to the invention for performing physiological measurements on a human body. Some examples of physiological signals originating from the human body are: electrophysiological signals, pressure, acidity (pH) and so on.

Device 1 comprises a first housing part 2 and a second housing part 3 which are shown in coupled position. In the shown preferred embodiment the first housing part 2 is provided with measuring means for measuring electrophysiological signals of a patient. An example of suitable measuring means are commercially obtainable adhesive electrodes. The electrodes (4A, 4B, 4C, 4D, 4E, 4F) are connected by means of electrical cables (5A, 5B, 5C, 5D, 5E, 5F) to first housing part 2. Six electrodes are shown by way of illustration. In practice the number of electrodes will vary in most cases between 2 and 24.

The second housing part 3 is provided with an electrode 6 for fixing device 1 on the body of a patient. Electrode 6 also serves as earth. A separate earth electrode (4G, 5G) can alternatively be provided.

For connection of the measuring means use is preferably made of shielded connecting cables (5A, 5B, 5C, 5D/, 5E, 5F, 5G) with a semiconducting coating. Suitable cables are commercially available.

FIG. 2 shows device 1 in partially cut-away view without measuring means. For power supply device 1 is provided with a solar cell 7 (FIG. 1) and (chargeable) batteries 8.

In order to bring about an electrical connection the first housing part 2 comprises an electrical connector 12 for co-action with an electrical connector 13 of second housing part 3.

FIG. 3 shows device 1 with exploded parts. The releasable coupling is brought about by means of co-acting edges 20 and 30 of the respective housing parts 2 and 3.

FIG. 4 shows a block diagram of the components of device 1 for the purpose of illustrating the operation thereof. After coupling of first housing part 2 and second housing part 3 identification means 21, 31 will determine for which type of electrophysiological measurement the coupled first housing part 2 is adapted. First housing part 2 is provided for this purpose with first identification means 21, which can be read by second identification means 31 in second housing part 3. The first identification means 21 comprise for instance an identification chip which defines the type of measurement for which the first housing part 2 is adapted (or the function of first housing part 2).

Identification means 31 inform processor 32 about the type of electrophysiological measurement associated with first housing part 2.

Communication means 33 serve to bring about wireless communication with a further device. Depending on the type of electrophysiological measurement, the appropriate associated software for processor 32 can for instance be retrieved from a remote computer.

The device 1 is now ready for use.

Measuring electrodes 4A-4F send measured electrophysiological signals via connectors 12 and 13 to amplifier means 35. Amplifier means 35 preferably comprise at least one reference amplifier. The reference amplifier is provided with N inputs to which all measuring means are connected. A shared reference connection carries the average measurement signal of all N connected measuring means. Via the N outputs of the reference amplifier N measurement signals are sent to processor 32 for processing. A reference amplifier suitable for this purpose has been designed by the inventor of the present device and is described in FIG. 3 of the article “A new technique for simultaneously recording EMG and movements in experimental animals”, Eykern L. A. van, Geisler, H. C., Gramsbergen, A. A., Brain Res Protoc. 6 (2001) (3): 108-118, this article being incorporated herein by reference.

Before the measurement signals reach processor 32 they are digitized by analog-digital converters 36.

Processor 32 has access to a memory 37 (ROM) in which, among other things, test means 34 are present for the purpose of testing the operation of all required measuring means 4A-4F. Test means 34 are preferably embodied in software. “Error” signals from measuring means which are not functioning or not functioning properly can be eliminated.

Processor 32 also has access to a writable storage medium 38 for storage of (some of) the processed measurement signals. The writable storage medium 38 is preferably removable, for instance in the form of a memory card. The processed measurement signals can optionally be stored in an external memory, whereafter the processed measurement signals can be transported by means of communication means 33.

The device according to the invention can be given an extremely compact form, for instance in the shown handheld embodiment, and is thereby suitable for ambulatory use by a patient.

The invention is based in a general sense on the insight of providing a measuring device for physiological signals which consists of two housing parts: one variable housing part with measuring means which is intended for a specific type of measurement, and one constant housing part which comprises the signal processing means for the measurement signals and which is suitable for any type of measurement. The variable housing part is highly suitable for once-only use, thereby guaranteeing the hygiene for every patient. In line with the inventive concept, an individual variable housing part with pre-connected measuring means is available for each type of measurement. The constant housing part is suitable and intended for co-action with all variable housing parts.

The device according to the invention is suitable for measuring physiological signals of an object generally. The device according to the invention is particularly suitable for measuring physiological signals of a human body. The device according to the invention is suitable for use in diverse medical fields. A few examples hereof are: gynaecology, gastro-enterology, urology, child neurology, lung function, intensive care, rehabilitation and movement sciences.

The invention is further not limited to the described and shown preferred embodiment, but extends to any embodiment falling within the scope of protection as defined in the claims, as seen in the light of the foregoing description and accompanying drawings.

1. A device for measuring physiological signals of an object, which device is provided with measuring means for measuring the physiological signals and with signal processing means for at least partially processing the measured physiological signals, characterized in that the device (1) comprises a first housing part (2) to which the measuring means (4A-4F) are attached, and that the device comprises a second housing part (3) in which the signal processing means are accommodated, wherein the housing parts are connectable by means of a releasable coupling.

2. A device as claimed in claim 1, wherein the measuring means comprise two or more electrodes (4A-4F) which are adapted to measure electrophysiological signals in a human body.

3. A device as claimed in claim 1, wherein the signal processing means comprise at least one reference amplifier.

4. A device as claimed in claim 1, wherein the releasable coupling comprises a snap connection (20, 30) for the housing parts (2, 3).

5. A device as claimed in claim 1, wherein the releasable coupling comprises an electrical connection (12, 13) for the measuring means (4A-4F) and the signal processing means.

6. A device as claimed in claim 1, wherein the second housing part (3) comprises test means (34) for testing the operation of the measuring means (4A-4F) of a coupled first housing part (2).

7. A device as claimed in claim 6, wherein the second housing part (3) comprises means (34) for switching off measuring means which are not functioning or not functioning properly.

8. A device as claimed in claim 1, wherein the first housing part (2) comprises first identification means with information relating to the type of physiological measurement for which the first housing part (2) is adapted, and wherein the second housing part (3) comprises second identification means (31) adapted to read the information.

9. A device as claimed in claim 1, wherein the second housing part (3) comprises means (33) for wireless communication with a further device.

10. A device as claimed in claim 9, wherein the second housing part (3) is adapted to retrieve software from a further device for the purpose of controlling one or more means (32) of the second housing part.
